(19) United States
(12) Patent Application Publication
(10) Pub. No.: US 2022/0023327 A1
(43) Pub. Date: Jan. 27, 2022

(54) MANGIFERIN-CONTAINING HERBAL COMPOSITIONS FOR IMPROVING SPORTS PERFORMANCE

(71) Applicant: NEKTIUM PHARMA, S.L., Agüimes - Las Palmas (ES)

(72) Inventors: Julia C. WIEBE, Las Palmas de GC (ES); Miguel JIMÉNEZ DEL RÍO, Telde (ES); Nigel GERICKE, Cape Town (ZA); Jose CALBET, Telde (ES)

(21) Appl. No.: 17/311,445

(22) PCT Filed: Dec. 7, 2018
(86) PCT No.: PCT/EP2018/084023

§ 371 Date: Jun. 7, 2021

(51) Int. Cl. A61K 31/7048; A61K 31/352; A61K 36/8905; A61K 36/48; A61K 36/489

(52) U.S. Cl. CPC A61K 31/7048; A61K 31/352; A61K 36/489; A61K 36/48; A61K 36/8905

(57) ABSTRACT

The present invention refers to a formulation for increasing sports performance and to a method for increasing sports performance, comprising administering said formulation to an athlete.

### FIELD OF THE INVENTION

The present invention relates to mangiferin-containing herbal formulations which improve performance by an athlete, sports person, or exerciser during exertion, by increasing peak power output by the athlete, sports person, or exerciser; mean power output by the athlete, sports person, or exerciser; tissue oxygenation in the athlete, sports person, or exerciser, or peak oxygen consumption by the athlete, sports person, or exerciser.

### BACKGROUND

Fatigue is a complex process which may originate in any structure intervening in the production and control of muscle contractions. Performance-enhancing compounds may exert their effects by facilitating energy supply and utilization, easing central command and motor control and reducing the negative effects caused by energy depletion, shortage of O2, metabolite accumulation, and reactive oxygen and nitrogen species (RONS) on force generation, muscle contraction activation and afferent feedback. Polyphenols are believed to have sports performance-enhancing properties. Polyphenols may act as antioxidants, signaling molecules, or hold anti-inflammatory, anti-aging, neuromodulatory or neuroprotective properties, which may confer their ergogenic potential. Most of these effects have only been demonstrated in cell culture or high-dose animal models.

Reactive oxygen and nitrogen radicals form during sprint exercise, with iron-catalyzed formation of hydroxyl radicals being accelerated by acidification from high glycolytic rates attained during sprints. Acidosis accelerates hydroxyl radical production and reduces the activities of antioxidant enzymes. Compounds which mitigate formation of hydroxyl radicals may improve performance during exercise.

The ergogenic potential of the polyphenols luteolin and mangiferin remains unknown, and the effects of quercetin on performance during repeated all-out prolonged sprints is yet to be studied in humans. Mangiferin (2-β-D-glucopyranosyl-1,3,6,7-tetrahydroxyxanthone) is a non-flavonoid polyphenol, present in mango leaves and other plants. Mangiferin protects against free radical production due to its iron-chelating properties. Mangiferin can traverse the blood-brain barrier and modulate neurotransmission. It remains unknown whether mangiferin attenuates the effects of ischemia/reperfusion in humans.

Quercetin is a flavonoid polyphenol found in several fruits and vegetables, including mangoes. Although quercetin has a low bioavailability due to its poor intestinal absorption, this may be improved by an oleaginous vehicle, such as tiger nut extract, which is rich in glyceryl esters of fatty acids. Quercetin, like mangiferin, is a phytoestrogen, capable of activating estrogen receptors.

Luteolin (30, 40, 50, 70-tetrahydroxyflavone) is a flavone and, like mangiferin and quercetin, is a potent antioxidant and inhibitor of xanthine oxidase. Luteolin is also a NADPH (nicotinamide adenine dinucleotide phosphate) oxidase inhibitor.

No study to date has determined the efficacy of natural polyphenols in mitigating the deterioration of skeletal muscle contractile function after short ischemia/reperfusion in humans. An object disclosed herein relates to use of mangiferin, administered with quercetin, tiger nut extract, and/or luteolin, to provide a performance-enhancing effect in men and women during exercise or physical exertion. An object disclosed herein relates to use of herbal formulations comprising mangiferin to mitigate ischemia/reperfusion injuries to muscle tissue during exertion.

The objects are illustrative of those that can be achieved by various embodiments disclosed herein, and are not intended to be limit the possible advantages which can be realized. Thus, these and other objects and advantages of the various exemplary embodiments will be apparent from the description herein or can be learned from practicing the disclosed embodiments, both as described herein or as modified in view of any variation that may be apparent to those skilled in the art. Accordingly, the present invention resides in the novel methods, arrangements, combinations, and improvements herein shown and described.

### BRIEF DESCRIPTION OF THE INVENTION

In light of the present need for safe methods of improving athletic performance, a brief summary of various exemplary embodiments is presented. Some simplifications and omissions may be made in the following summary, which is intended to highlight and introduce some aspects of the various exemplary embodiments, but not to limit the scope of the invention. Detailed descriptions of a preferred exemplary embodiment adequate to allow those of ordinary skill in the art to make and use the inventive concepts will follow in later sections.

Various embodiments disclosed herein relate to a formulation for increasing sports performance, comprising at least one herbal ingredient. In various embodiments, the formulation comprises from 25 mg to 5,000 mg, from 25 mg to 3,000 mg, from 25 mg to 2,000 mg, from 35 mg to 1,500 mg, from 55 mg to 1,000 mg, from 65 mg to 500 mg, from 75 mg to 250 mg, or from 84 mg to 140 mg of mangiferin. The mangiferin may be administered as substantially pure mangiferin, where substantially pure mangiferin is pharmaceutically acceptable and contains >80% by weight mangiferin; >90% by weight mangiferin; >95% by weight mangiferin; or >99% by weight mangiferin. The mangiferin may be administered as a component of a plant extract, wherein the plant extract comprises from 10% to 90% by weight mangiferin; from 20% to 85% by weight mangiferin; from 40% to 75% by weight mangiferin; or from 50% to 70% by weight mangiferin, or about 60% by weight mangiferin. In various embodiments, the mangiferin is administered as a component of a mango leaf extract. The mangiferin may be a component of an extract obtained by extraction of mango leaves with water, a polar protic solvent, or a polar aprotic solvent. The extract may be obtained by extraction of mango leaves with water or a lower alcohol.

According to various embodiments disclosed herein, mangiferin may be administered in combination with a second herbal active ingredient. This second herbal active ingredient may be luteolin in an amount of between 10 mg and 5,000 mg, between 20 mg and 4,000 mg, between 30 mg and 2,000 mg, between 45 mg to 1,000 mg, or between 50 mg to 500 mg per day. In various embodiments, the luteolin is administered as a component of an Arachis hypogeae or Perilla frutescens extract. The luteolin may be a component of an extract obtained by extraction of Arachis hypogeae or Perilla frutescens with water, a polar protic solvent, a polar aprotic solvent, a nonpolar solvent, or mixtures thereof. The extract may be obtained by extraction of Arachis hypogeae or Perilla frutescens with a lower alcohol, ethyl acetate, a hydrocarbon solvent, or a halogenated hydrocarbon solvent.

In some embodiments disclosed herein, mangiferin may be administered in combination with quercetin. Quercetin may be administered in an amount of between 50 mg and 10,000 mg, between 100 mg and 8,000 mg, between 150 mg and 6,000 mg, between 300 mg and 4,000 mg, or between 500 mg and 2,000 mg per day. In various embodiments, the quercetin is administered as a component of a Sophora japonica extract. The quercetin may be a component of an extract obtained by extraction of Sophora japonica with water, a polar protic solvent, a polar aprotic solvent, or mixtures thereof. The extract may be obtained by extraction of Sophora japonica with water, a lower alcohol, a mixture of water and a C1-C4 alcohol, or ethyl acetate.

In further embodiments, mangiferin may be administered in combination with a high potency fraction of Cyperus esculentus tubers. The high potency fraction of Cyperus esculentus tubers is obtained by extraction with ethyl acetate to obtain an organic solvent soluble fraction. The high potency fraction comprises:

from 70% to 95% by weight oleic acid glyceryl esters, from 80% to 94% by weight oleic acid glyceryl esters, from 85% to 93% by weight oleic acid glyceryl esters, from 90% to 92% by weight oleic acid glyceryl esters, or about 91% by weight oleic acid glyceryl esters;

from 1% to 15% by weight linoleic acid glyceryl esters, from 3% to 14% by weight linoleic acid glyceryl esters, from 5% to 12% by weight linoleic acid glyceryl esters, from 6% to 9% by weight linoleic acid glyceryl esters, or about 7% by weight linoleic acid glyceryl esters; phytosterols, such as stigmasterol, in an amount of 0.2% by weight or more; and

flavonoids, such as myricetin, in an amount of 0.2% by weight or more.

The high potency fraction of Cyperus esculentus tubers is administered in an amount of between 5 mg and 5,000 mg per day; between 10 mg and 500 mg per day; or between 15 mg and 350 mg per day.

Various embodiments disclosed herein relate to a formulation for increasing sports performance, comprising mangiferin and at least one of luteolin in an amount of between 10 mg and 5,000 mg, quercetin in an amount of between 50 mg and 10,000 mg; and a high potency fraction of Cyperus esculentus tubers in an amount of between 5 mg and 5,000 mg per day from 100 mg to 10 g quercetin; from 50 mg to 5,000 mg of an ethyl acetate extract of Cyperus esculentus tubers; and mixtures thereof. In various embodiments, the formulation comprises from 50 mg to 5,000 mg of mangiferin and from 10 mg to 5,000 mg of luteolin. In some embodiments, the formulation comprises from 50 mg to 5,000 mg of mangiferin; from 100 mg to 10 g quercetin; and from 50 mg to 5,000 mg of an ethyl acetate extract of Cyperus esculentus tubers.

In various embodiments disclosed herein, the mangiferin formulation comprises a single dosage form for once-daily administration. In certain embodiments, the formulation comprises multiple dosage forms, wherein each dosage form has similar contents, allowing a desired daily dosage to be administered in multiple divided doses. In some embodiments, the formulation comprises a first dosage form comprising mangiferin; and a second dosage form comprising luteolin, quercetin, an ethyl acetate extract of Cyperus esculentus tubers, or a mixture thereof.

Various embodiments disclosed herein relate to methods for increasing performance by a person engaged in physical activity, e.g., physical exercise, an individual sport, or a team sport. Such a person, here referred to as an athlete, a sports person, or an exerciser, may be administered from 50 mg to 5,000 mg of mangiferin. The mangiferin may be administered as a sole component, or the mangiferin may be administered in combination with at least one active ingredient selected from the group consisting of luteolin; quercetin; and an ethyl acetate extract of Cyperus esculentus tubers; and mixtures thereof. The formulation increases sports performance in a male or female athlete by increasing peak power output by the athlete during physical exertion, e.g., running, cycling, or swimming; by increasing mean power output by the male or female athlete during physical exertion; by increasing brain frontal lobe oxygenation by a female athlete during physical exertion; and/or by increasing peak oxygen consumption by the female athlete.

Certain embodiments disclosed herein relate to methods for increasing sports performance by increasing power output during physical exertion by a male or female athlete, by administering a combination of mangiferin and luteolin to the athlete. The combination is administered in a single daily dosage, or from two to five divided doses per day. The total daily dosage is:

from 25 mg to 5,000 mg, from 25 mg to 3,000 mg, from 25 mg to 2,000 mg, from 35 mg to 1,500 mg, from 55 mg to 1,000 mg, from 65 mg to 500 mg, from 75 mg to 250 mg, or from 84 mg to 140 mg of mangiferin per day; and from 10 mg and 5,000 mg, from 20 mg and 4,000 mg, from 30 mg and 2,000 mg, from 45 mg to 1,000 mg, or from 50 mg to 500 mg luteolin per day.

Certain embodiments disclosed herein relate to methods for increasing sports performance by increasing power output during physical exertion by a male or female athlete, by administering a combination of mangiferin, quercetin, and an ethyl acetate extract of Cyperus esculentus tubers to the athlete. The combination is administered in a single daily dosage, or from two to five divided doses per day. The total daily dosage is:

from 25 mg to 5,000 mg, from 25 mg to 3,000 mg, from 25 mg to 2,000 mg, from 35 mg to 1,500 mg, from 55 mg to 1,000 mg, from 65 mg to 500 mg, from 75 mg to 250 mg, or from 84 mg to 140 mg of mangiferin per day;

between 50 mg and 10,000 mg, between 100 mg and 8,000 mg, between 150 mg and 6,000 mg, between 300 mg and 4,000 mg, or between 500 mg and 2,000 mg of quercetin per day; and between 5 mg and 5,000 mg; between 10 mg and 500 mg per day; or between 15 mg and 350 mg per day of a high potency fraction of Cyperus esculentus tubers.

Various embodiments described herein relate to methods for increasing sports performance by increasing brain oxygenation, preventing fatigue, and/or increasing peak oxygen consumption during physical exertion by a female athlete, by administering a mangiferin composition to the athlete. The combination is administered in a single daily dosage, or from two to five divided doses per day. The total daily dosage is:

from 25 mg to 5,000 mg, from 25 mg to 3,000 mg, from 25 mg to 2,000 mg, from 35 mg to 1,500 mg, from 55 mg to 1,000 mg, from 65 mg to 500 mg, from 75 mg to 250 mg, or from 84 mg to 140 mg of mangiferin per day; and at least one of

from 10 mg and 5,000 mg, from 20 mg and 4,000 mg, from 30 mg and 2,000 mg, from 45 mg to 1,000 mg, or from 50 mg to 500 mg luteolin per day;

between 50 mg and 10,000 mg, between 100 mg and 8,000 mg, between 150 mg and 6,000 mg, between 300 mg and 4,000 mg, or between 500 mg and 2,000 mg of quercetin per day; and

between 5 mg and 5,000 mg; between 10 mg and 500 mg per day; or

between 15 mg and 350 mg per day of a high potency fraction of Cyperus esculentus tubers.

### FIGURES

FIG. 1: Shows the experimental protocol for measuring the influence of mangiferin extracts formulations on sports performance.

FIG. 2: Shows peak power output (Wpeak) observed during the experimental protocol of FIG. 1.

FIG. 3: Shows the mean power output (Wmean) observed during the experimental protocol of FIG. 1.

FIG. 4: Shows brain oxygenation (Frontal lobe tissue oxygenation index: TOI (%)) observed during the experimental protocol of FIG. 1. Dashed lines represent the values recorded at rest.

FIG. 5: Shows vastus lateralis Oxygenation Index (%) Observed During the experimental protocol of FIG. 1. Dashed lines represent the values recorded at rest. Dotted lines represent the values observed during the last 5 s of ischemia after sprint 3, i.e., the value corresponding to “zero oxygenation.”

FIG. 6: Experimental protocol for measuring the influence of mangiferin and luteolin extracts formulations on sports performance.

FIG. 7: Performance during the sprint exercise after the ingestion of polyphenols (luteolin+mangiferin) or placebo. A) Peak power output in 15 s sprints performed after ischemia. B) Mean power output during the first 5 s during the sprints performed after ischemia. SIE: first sprint after incremental exercise, SSIE: second sprint after incremental exercise. Number 1 indicates after 48 h and 2 after 15 days of supplementation. C) Mean power output during the 30 s Wingate test. WG: Wingate test, the first number represents the Wingate order number (1, 2 or 3), the second number (1 or 2) indicates after 48 h and 2 after 15 days of supplementation, respectively. * P<0.05 compared with 48 h test in the same condition. $ P<0.05 for treatment effect. ANOVA Wingate×time×treatment P=0.027). N=12.

FIG. 8: Frontal lobe oxygenation index (TOI) during the first two 30 s Wingate tests after the ingestion of polyphenols (luteolin+mangiferin) or placebo. Number 1 indicates after 48 h and 2 after 15 days of supplementation. $ P<0.05 for treatment effect. N=12.

FIG. 9: Quadriceps muscle oxygenation index (TOI, mean of the Musculus vastus lateralis and Medialis (%)) during the first two 30 s Wingate tests after the ingestion of polyphenols (luteolin+mangiferin) or placebo. Number 1 indicates after 48 h and 2 after 15 days of supplementation. $ P<0.05 for treatment effect. N=12.

### DETAILED DESCRIPTION OF THE INVENTION

The term “athlete,” as used herein, generally relates to any person engaged in physical exercise, performing in an individual sport, or participating in a team sport. The terms “athlete,” “sports person,” and “exerciser,” unless otherwise specified, should be understood to be synonymous for the purpose of this disclosure.

Various embodiments disclosed herein relate to supplements containing a mango leaf extract rich in mangiferin. These supplements enhance performance in humans during high intensity exercise. In various embodiments, the supplements comprise a first component from 25 mg to 5,000 mg, from 25 mg to 3,000 mg, from 25 mg to 2,000 mg, from 35 mg to 1,500 mg, from 55 mg to 1,000 mg, from 65 mg to 500 mg, from 75 mg to 250 mg, or from 84 mg to 140 mg of mangiferin, administered per day in a single dose or multiple divided doses. The mangiferin may be administered as substantially pure mangiferin; or as a component of a plant extract, wherein the plant extract comprises from 10% to 90% by weight mangiferin; from 20% to 85% by weight mangiferin; from 40% to 75% by weight mangiferin; or from 50% to 70% by weight mangiferin, or about 60% by weight mangiferin. In various embodiments, the mangiferin is administered as a component of a mango leaf extract comprising 60% or more of mangiferin; up to 2.5% of isomangiferin; trace levels of isomangiferin; and up to 10% of sugars, based on weight %.

According to various embodiments disclosed herein, mangiferin may be administered in combination with a second herbal active ingredient. This second herbal active ingredient may comprise luteolin in an amount of between 10 mg and 5,000 mg, between 20 mg and 4,000 mg, between 30 mg and 2,000 mg, between 45 mg to 1,000 mg, or between 50 mg to 500 mg per day. In various embodiments, the second herbal active ingredient is an extract of Arachis hypogeae shells or Perilla frutescens herb, comprising at least 90% by weight luteolin. Alternatively, the second herbal active ingredient may comprise quercetin. Quercetin may be administered in an amount of between 50 mg and 10,000 mg, between 100 mg and 8,000 mg, between 150 mg and 6,000 mg, between 300 mg and 4,000 mg, or between 500 mg and 2,000 mg per day. In various embodiments, the second herbal active ingredient is a Sophora japonica extract, comprising at least 90% by weight quercetin. In further embodiments, mangiferin may be administered in combination with a high potency fraction of Cyperus esculentus tubers as a second herbal active ingredient. The high potency fraction of Cyperus esculentus tubers is obtained by extraction with ethyl acetate to obtain an organic solvent soluble fraction.

The high potency fraction comprises:

from 70% to 95% by weight oleic acid glyceryl esters, from 80% to 94% by weight oleic acid glyceryl esters, from 85% to 93% by weight oleic acid glyceryl esters, from 90% to 92% by weight oleic acid glyceryl esters, or about 91% by weight oleic acid glyceryl esters;

from 1% to 15% by weight linoleic acid glyceryl esters, from 3% to 14% by weight linoleic acid glyceryl esters, from 5% to 12% by weight linoleic acid glyceryl esters, from 6% to 9% by weight linoleic acid glyceryl esters, or about 7% by weight linoleic acid glyceryl esters; phytosterols, such as stigmasterol, in an amount of 0.2% by weight or more; and

flavonoids, such as myricetin, in an amount of 0.2% by weight or more. Various embodiments disclosed herein relate to supplements comprising from 25 mg to 5,000 mg, from 25 mg to 3,000 mg, from 25 mg to 2,000 mg, from 35 mg to 1,500 mg, from 55 mg to 1,000 mg, from 65 mg to 500 mg, from 75 mg to 250 mg, or from 84 mg to 140 mg of mangiferin; and between 10 mg and 5,000 mg, 20 mg and 4,000 mg, 30 mg and 2,000 mg, 45 mg to 1,000 mg, 50 mg to 500 mg luteolin, or 50 mg to 150 mg luteolin, in a single dosage form or multiple divided dosage forms. In various embodiments, the supplements comprise from 65 mg to 500 mg, from 75 mg to 250 mg, or about 140 mg of mango leaf extract comprising 60% mangiferin; and from 50 mg to 150 mg luteolin, in a single dosage form or in divided doses.

In various embodiments, the present disclosure describes supplements comprising:
from 25 mg to 5,000 mg, from 25 mg to 3,000 mg, from 25 mg to 2,000 mg, from 35 mg to 1,500 mg, from 55 mg to 1,000 mg, from 65 mg to 500 mg, from 75 mg to 250 mg, or from 84 mg to 140 mg of mangiferin per day;
between 50 mg and 10,000 mg, between 100 mg and 8,000 mg, between 150 mg and 6,000 mg, between 300 mg and 4,000 mg, or between 500 mg and 2,000 mg of quercetin per day; and.
a high potency ethyl acetate extract of Cyperus esculentus tubers in an amount of from 17 mg/day to 350 mg/day.

In various embodiments, supplements comprising mangiferin in combination with luteolin or a mixture of quercetin and a high potency ethyl acetate extract of Cyperus esculentus increase peak power output after ischemia of a skeletal muscle, followed by reperfusion. This effect is seen in both men and women. In women, mangiferin supplements improve brain oxygenation at rest and during exercise, and increased peak VO₂ during high-intensity exercise. Mangiferin extracts enhance performance during physical exertion, without leading to significant increases in consumption of oxygen. Moreover, a trend for better muscular extraction of O₂ was observed during physical exertion performed after ischemia/reperfusion when the subjects had taken the combined MLE/quercetin/tiger.

Two supplements containing MLE had positive effects on performance during physical exertion. However, mangiferin/quercetin/tiger nut extract combinations are superior to mangiferin/luteolin combinations, particularly regarding the effects on power output during exercise following ischemia and reperfusion of skeletal muscles. While luteolin attenuates the ischemia/reperfusion injury in several tissues, it remains unknown whether luteolin prevents ischemia/reperfusion injury in skeletal muscle. Quercetin may protect skeletal muscle from ischemia/reperfusion injury in rodents submitted to ischemia. However, quercetin supplementation for 1 week in 30 m running sprints has been reported to reduce athletic performance. The present data show that an increase in performance, measured in terms of mean and peak power output during physical activity, was observed when mangiferin was administered, suggesting that mangiferin may be responsible for the effect on power output.

In various embodiments disclosed herein, administration of mangiferin in combination with luteolin or quercetin does not increase blood lactate responses or carbohydrate oxidation during submaximal exercise or other physical exertion. Although mangiferin activates pyruvate dehydrogenase (PDH) in cell cultures, resulting in reduced lactate production and increase carbohydrate oxidation, changes in lactate and carbohydrate levels during exercise were not observed when mangiferin was combined with luteolin or quercetin.

Muscle energy efficiency is reduced during high intensity exercise by several mechanisms which include, among others, increased recruitment of less efficient type II muscle fibers, lactic acidosis, electrolyte alterations, and the generation of reactive oxygen and nitrogen species (RONS). During high intensity exercise, RONS are produced due to both the high mitochondrial respiratory rate and the activation of the anaerobic metabolism. RONS may contribute to muscle fatigue by reducing calcium sensitivity, and reducing calcium release from sarcoplasmic reticulum. Mangiferin supplements may enhances myofilament Ca²⁺ sensitivity, which may result in greater force production if the required energy is available.

Excessive RONS production could reduce mitochondrial phosphate/oxygen ratio, or P/O ratio, while antioxidants in mangiferin supplements may favorably influence mitochondrial increase the P/O ratio. Moreover, the ingestion of antioxidants before physical exertion reduces the level of protein carbonyls in muscle and plasma, and lowers the glycolytic rate without a detrimental effect on performance.

Various embodiments disclosed herein relate to use of the polyphenols mangiferin, luteolin, quercetin, and combinations thereof for quenching free radicals generated during exercise or physical exertion. The three polyphenols discussed herein also inhibit xanthine oxidase. The present disclosure shows for the first time that antioxidants are capable of enhancing peak power output and mean power output during the fatigued state induced by repeated prolonged sprint exercise. These compounds thus enhance performance during sports activity or manual labor.

The antioxidant properties of the polyphenol supplements described herein may contribute to enhanced physical performance. However, a wide variety of antioxidants have previously failed to enhance peak power output in humans, and none have shown these properties in the fatigued state. To boost performance in a fatigued muscle, greater calcium release is needed to enhance the number of cross-bridges of muscle filaments that can be established, but also a faster calcium reuptake is required to shorten the relaxation phase. Caffeine can enhance force in fatigued muscle by boosting Ca2+ release, but the dose needed to cause a significant change in performance would be lethal for humans. Mangiferin, a major component of mango leaf extract, shares some common intracellular mechanisms of action with caffeine, which may facilitate calcium release in the fatigued state (i.e., when Ca2+ release is depressed). Like caffeine and beta-agonists, mangiferin increases cyclic AMP (cAMP) levels, and can, through the activation of protein kinase A (PKA), stimulate slow-twitch skeletal muscle isoform (SERCA) activity. However, at tolerable doses, caffeine does not alter skeletal muscle metabolism. The main mechanism of the ergogenic action of caffeine is its effect on the central nervous system, by enhancing muscle activation.

Although caffeine may enhance performance during prolonged exercise and team-sport activities, caffeine is unlikely to enhance power and strength under normal use. Moreover, there is no evidence supporting an ergogenic effect of caffeine during episodes of ischemia/reperfusion in sport disciplines. Unlike caffeine, which may cause hypokalemia in athletes, mangiferin/luteolin and mangiferin/quercetin extracts cause no significant changes during physical exertion on plasma calcium, potassium, sodium, and chloride levels.

Reduction in brain oxygenation is associated with fatigue. Moreover, at exhaustion during exercise improving the oxygenation of the brain and upper body by increasing oxygen intake while maintaining the lower extremities in a deoxygenated state by occluding circulation, was associated with improved performance. This supports a mechanistic link between brain oxygenation and fatigue during sprint exercise in a fatigued state. In various embodiments disclosed herein, mangiferin-containing supplements consumed before sprint exercise may counteract fatigue in female athletes by improving brain oxygenation.

The present disclosure describes the protective effects of a polyphenol combination including mango leaf extract (MLE), quercetin, and tiger nut extract on functional deterioration induced by an inadequate blood supply to muscle tissue (ischemia), followed by reperfusion of blood into the muscle tissue.

The results presented herein show that mangiferin-containing MLE has a remarkable ergogenic effect increasing muscle power in fatigued subjects, without increasing oxygen consumption, submaximal exercise efficiency, or submaximal and maximal blood lactate concentrations. This is expected for a compound acting on the central nervous system. MLE, when combined with quercetin and tiger nut extract, assists in maintaining skeletal muscle function during ischemia/reperfusion, strongly suggesting that this combination is also acting directly on the skeletal muscles.

The terms “effective amount” or “dose” as used herein are interchangeable and may refer to the amount of an active ingredient or agent or composition that elicits a biological response in a tissue, system, animal, individual or human that is being sought by a researcher, veterinarian, medical doctor or other clinician, or any combination thereof. A biological or response may include, for example, the following: (1) increasing sports performance.

In the context of the present invention the term “part” in relation with the formulation refers to the amount and/or ratio in mass of each of the ingredients of said formulation.

In the context of the present invention the expression “acute phase” refers to the phase of about 48 hours of supplementation.

In the context of the present invention the expression “chronic phase” to the phase of about two weeks hours of supplementation.

In the context of the present invention the expression “carrier” refers to forms to which substances are incorporated to improve the delivery and the effectiveness of formulations or drugs. Carriers are used in drug-delivery systems such as the controlled-release technology to prolong in vivo drug actions, decrease drug metabolism, and reduce drug toxicity. Carriers are also used in designs to increase the effectiveness of drug delivery to the target sites of pharmacological actions (U.S. National Library of Medicine. National Institutes of Health). Adjuvant is a substance added to a drug product formulation that affects the action of the active ingredient in a predictable way. Vehicle is an excipient or a substance, preferably without therapeutic action, used as a medium to give bulk for the administration of medicines (Stedman's Medical Spellchecker, © 2006 Lippincott Williams & Wilkins). Such pharmaceutical carriers, adjuvants or vehicles can be sterile liquids, such as water and oils, including those of petroleum, animal, vegetable or synthetic origin, such as peanut oil, soybean oil, mineral oil, sesame oil and the like, excipients, disgregants, wetting agents or diluents. Suitable pharmaceutical carriers are described in “Remington's Pharmaceutical Sciences” by E. W. Martin. The selection of these excipients and the amounts to be used will depend on the form of application of the pharmaceutical composition.

### EMBODIMENTS

1) A particular embodiment is directed to a formulation for increasing sports performance, comprising:
   a. an effective amount of mangiferin; in combination with
   b. an effective amount of an active ingredient selected from the group consisting of luteolin, quercetin, an ethyl acetate extract of Cyperus esculentus tubers and mixtures thereof.
2) The formulation according to embodiment 1; wherein the formulation comprises:
   from 5 parts to 1000 parts of mangiferin, in combination with either:
      from 2 to 1000 parts of luteolin,
      from 20 to 2000 parts of quercetin,
      from 1 to 1000 parts of an ethyl acetate extract of Cyperus esculentus tubers;
      or with mixtures thereof.
3) The formulation according to any of the previous embodiments, wherein the formulation comprises from 10 to 1000 parts of mangiferin and from 2 to 1000 parts of luteolin.
4) The formulation according to any of the previous embodiments, wherein the formulation comprises from 10 to 1000 parts of mangiferin; from 20 to 2000 parts of quercetin; and from 1 to 1000 parts of an ethyl acetate extract of Cyperus esculentus tubers.
5) The formulation according to embodiments 1 and 2, comprising:
   a. from 25 mg to 5,000 mg of mangiferin; in combination with either:
      from 10 mg to 5,000 mg of luteolin,
      from 100 mg to 10 g quercetin,
      from 5 mg to 5,000 mg of an ethyl acetate extract of Cyperus esculentus tubers,
or with mixtures thereof.
6) The formulation according to any of the previous embodiments, wherein the formulation comprises a single dosage form.
7) The formulation according to any of the previous embodiments, wherein the formulation comprises multiple dosage forms.
8) The formulation according to embodiment 7, wherein the formulation comprises multiple dosage forms, wherein each dosage form has similar contents.
9) The formulation according to embodiment 7, wherein the formulation comprises multiple dosage forms, wherein a first dosage form comprises said mangiferin and a second dosage form comprises said either luteolin, quercetin, an ethyl acetate extract of Cyperus esculentus tubers or mixtures thereof.
10) The formulation according to any of the previous embodiments, wherein
   said mangiferin is a component of an extract of Mangifera indica, said extract of Mangifera indica comprising from 10% to 90% by weight of mangiferin.
11) The formulation according to any of the previous embodiments, wherein
   said luteolin is a component of an extract of Arachis hypogaea or Perilla frutescens, said extract of Arachis hypogaea or Perilla frutescens comprising from 50% to 95% by weight of luteolin.
12) The formulation according to any of the previous embodiments, wherein
   said quercetin is a component of an extract of Sophora japonica, said extract of Sophora japonica comprising from 50% to 95% by weight of quercetin.
13) The formulation according to any of the previous embodiments, wherein said ethyl acetate extract of Cyperus esculentus tubers comprises:
   oleic acid glyceryl esters, linoleic acid glyceryl esters, or a combination thereof;
      phytosterols;
      flavonoids; and
      mixtures thereof.
14) A particular embodiment is directed to a method for increasing sports performance, comprising administering a formulation to an athlete, said formulation comprising:
   a. an effective amount of mangiferin; in combination with an effective amount of an active ingredient selected from the group consisting of luteolin, quercetin, an ethyl acetate extract of Cyperus esculentus tubers, and mixtures thereof; and
   b. optionally, a carrier;
      wherein increasing sports performance comprises at least one of:
   increasing power output by said athlete;
   increasing brain frontal lobe oxygenation or peak oxygen consumption in said athlete;
   mitigating the effects of ischemia/reperfusion injury in skeletal muscle.
15) The method for increasing sports performance according to embodiment 14, comprising administering a formulation to an athlete, said formulation comprising:
   from 5 parts to 1000 parts/day of mangiferin; in combination with either:
      from 2 to 1000 parts of luteolin,
         from 20 to 2000 parts of quercetin,
         from 1 to 1000 parts of an ethyl acetate extract of Cyperus esculentus tubers;
      or with mixtures thereof; and
      optionally, a carrier;
      wherein increasing sports performance comprises at least one of:
         increasing power output by said athlete;
         increasing brain frontal lobe oxygenation or peak oxygen consumption in said athlete;
         mitigating the effects of ischemia/reperfusion injury in skeletal muscle.
16) The method for increasing sports performance according to any of embodiments 14 and 15, comprising administering a formulation to an athlete, said formulation comprising:
   from 50 mg to 5,000 mg/day of mangiferin; in combination with either:
      from 10 mg/day to 5,000 mg/day of luteolin;
      from 100 mg/day to 10 g/day quercetin;
      from 50 mg/day to 5,000 mg/day of an ethyl acetate extract of Cyperus esculentus tubers;
   or mixtures thereof; and
      optionally, a carrier;
      wherein increasing sports performance comprises at least one of:
   increasing power output by said athlete;
   increasing brain frontal lobe oxygenation or peak oxygen consumption in said athlete;
   mitigating the effects of ischemia/reperfusion injury in skeletal muscle.
17) The method for increasing sports performance according to any of embodiments 14 to 16, wherein the said athlete is a woman.
18) The method according to any of embodiments 14-17, wherein increasing sports performance comprises increasing power output by said athlete, wherein power output is measured in terms of peak power output or mean power output.
19) The method according to any of embodiments 14-18, wherein increasing sports performance comprises increasing brain frontal lobe oxygenation or peak oxygen consumption in a female athlete.
20) The method according to any of embodiments 14-19, wherein increasing sports performance comprises mitigating the effects of ischemia/reperfusion injury in skeletal muscle.
21) The method of embodiment 12, wherein the formulation comprises from 50 mg/day to 5,000 mg/day of mangiferin and from 10 mg/day to 5,000 mg/day of luteolin.
22) The method according to any of embodiments 14-21, wherein the formulation comprises from 50 mg/day to 5,000 mg/day of mangiferin; from 100 mg/day to 10 g/day quercetin; and from 5 mg/day to 5,000 mg/day of an ethyl acetate extract of Cyperus esculentus tubers.
23) The method according to any of embodiments 14-22, wherein said mangiferin is a component of an extract of Mangifera indica, said extract of Mangifera indica comprising from 10% to 90% by weight of mangiferin
24) The method according to any of embodiments 14-23, wherein said increasing sports performance is observed for the acute and chronic phases.
25) The method according to any of embodiments 14-24, wherein said luteolin is a component of an extract of Arachis hypogaea, said extract of Arachis hypogaea comprising from 50% to 95% by weight of luteolin.
26) The method according to any of embodiments 14-25, wherein:
   said quercetin is a component of an extract of Sophora japonica, said extract of Sophora japonica comprising from 50% to 95% by weight of quercetin; and
   said ethyl acetate extract of Cyperus esculentus tubers comprises:
      oleic acid glyceryl esters, linoleic acid glyceryl esters, or a combination thereof;
      phytosterols;
      flavonoids; and
      mixtures thereof.
27) The formulation according to any of embodiments 1-13, wherein the formulation comprises from 50 mg to 5,000 mg of mangiferin and from 10 mg to 5,000 mg of luteolin.
28) The formulation according to any of embodiments 1-13, wherein the formulation comprises from 50 mg to 5,000 mg of mangiferin; from 100 mg to 10 g quercetin; and from 5 mg to 5,000 mg of an ethyl acetate extract of Cyperus esculentus tubers.
29) The formulation according to any of embodiments 1-13 and 27-28, wherein the formulation further comprises a carrier.

### EXAMPLES

The present invention will now be described by way of examples which serve to illustrate the construction and testing of illustrative embodiments. However, it is understood that the present invention is not limited in any way to the examples below.

### Example 1. Studies on Human Patients: Experimental Procedure

A. Subjects

Data on the effect of mangiferin-containing compositions was obtained from 17 men and 13 women. Subjects were requested to avoid strenuous exercise 48 h before the laboratory test and not to drink beverages containing caffeine or taurine during the 24 h preceding the test.

TABLE 1
Wmax, maximal intensity during the incremental exercise
test to exhaustion; Wpeak₁, instantaneous peak power
output during the Wingate test; LLM, lean mass of the lower
extremities; Wmean, mean power output during a 30 s
Wingate test; Accumulated VO₂, total amount of
O₂ consumed); % Anaerobic Energy, percentage
of the energy obtained through anaerobic pathways.

| | Men (n = 17) | Women (13) | P |
|---|---|---|---|
| Age (years) | 22.7 ± 2.1 | 27.0 ± 2.2 | 0.005 |
| Height (cm) | 176.9 ± 4.2 | 164.4 ± 4.6 | 0.000 |
| Weight (kg) | 71.2 ± 5.2 | 56.5 ± 5.4 | 0.000 |
| % body fat | 18.4 ± 3.7 | 26.0 ± 4.9 | 0.000 |
| Lean; mass of both legs (kg) | 19.8 ± 2.0 | 13.6 ± 2.5 | 0.000 |
| Hemoglobin (g · dL⁻¹) | 15.0 ± 0.8 | 13.2 ± 0.9 | 0.000 |
| HRmax (Beats/min) | 191.7 ± 7.5 | 189.3 ± 0.7 | 0.567 |
| VO₂max (mL/kg/min) | 47.5 ± 6.1 | 41.2 ± 6.1 | 0.005 |
| VO₂max (mL/kg LLM/min) | 171.1 ± 16.3 | 170.4 ± 15.7 | 0.921 |
| Wmax (W) | 259.1 ± 32.7 | 177.7 ± 38.0 | 0.000 |

Constant-intensity test at 120% VO₂max

| | | | |
|---|---|---|---|
| Endurance time (s) | 150.4 ± 40.1 | 132.0 ± 40.3 | 0.168 |
| 120% VO₂max intensity (W) | 303.8 ± 36.6 | 216.6 ± 40.5 | 0.000 |
| Work (kj · kg⁻¹ LLM) | 2.30 ± 0.60 | 1.98 ± 0.60 | 0.086 |
| O₂ deficit (mL) | 3362 ± 839 | 1880 ± 848 | 0.000 |
| O₂ deficit (mL · kg⁻¹ BW) | 47.2 ± 11.6 | 33.4 ± 11.4 | 0.001 |
| O₂ deficit/LLM | 169.3 ± 35.9 | 137.9 ± 34.1 | 0.011 |
| % Anaerobic Energy | 33.6 ± 6.3 | 32.1 ± 5.8 | 0.527 |

30 s Wingate test

| | | | |
|---|---|---|---|
| Wpeakᵢ | 1087.1 ± 86.5 | 753.0 ± 93.4 | 0.000 |
| Wpeakᵢ/kg | 15.3 ± 1.2 | 13.4 ± 1.2 | 0.000 |
| Wpeakᵢ/LLM | 55.4 ± 6.0 | 55.3 ± 6.5 | 0.979 |
| Wmean | 628.0 ± 65.6 | 417.3 ± 77.0 | 0.000 |
| Wmean/kg | 8.8 ± 0.8 | 7.4 ± 0.9 | 0.000 |
| Wmean/kg LLM | 31.9 ± 3.1 | 30.7 ± 3.0 | 0.270 |

Body composition of the subjects was determined by dual-energy x-ray absorptiometry (Lunar iDXA, GE Healthcare, Wisconsin; USA). Subjects were tested to determine peak oxygen consumption (VO₂peak), maximal heart rate (HRmax) and maximal power output (Wmax) in normoxia (F₁O₂: 0.21, P₁O₂: 143 mmHg) with an incremental exercise test to exhaustion with verification. The test started with 3 min at 20 W, followed by 15 and 20 W increases every 3 min in women and men, respectively, until the respiratory exchange ratio (RER) was >1.0.

After completion of the intensity with an RER ≤1.0, the intensity was increased by 10 and 15 W/min increase (women and men, respectively) until exhaustion. The intensity attained at exhaustion was taken at the maximal power output of the incremental exercise test (Wmax). At exhaustion, the ergometer was unloaded and subjects remained seated on the cycle ergometer pedaling at a slow speed (30-40 rpm) for 3 min. Thereafter, the verification test started at Wmax+5 W for 1 min, followed by 4 and 5 W increase (women and men, respectively) every 20 s until exhaustion. Between 1 and 2 weeks later, subjects reported to the laboratory on two occasions separated by at least 1 week, to carry out a constant-intensity supramaximal exercise to exhaustion at 120% of VO2max. This test was used to determine the anaerobic capacity. The constant-intensity supramaximal exercise test with longer endurance time to exhaustion was retained as representative for each subject. Data on the test subjects is presented in Table 2.

B. Power Output, Oxygen Uptake, and Supramaximal Exercise O2 Demand and Deficit

Power output during the sprint was reported as instantaneous peak power output (Wpeak) and mean power output (Wmean) throughout the duration of the sprints. Oxygen uptake was measured with a metabolic cart, calibrated according with high-grade certified gases. Respiratory variables were analyzed breath-by-breath and averaged every 20 s during the incremental exercise tests and during the repeated sprints. The highest 20 s averaged VO₂ recorded during the incremental test (i.e., including the verification phase) was taken as VO₂peak. The O2 demand during the sprints was calculated from the linear relationship between the last 20 s averaged VO₂ of each load, from 80 W up to 80-90% of VO₂max, while subjects were pedaling at 80 rpm. The accumulated oxygen deficit (AOD), representing the difference between O₂ demand and VO₂, was determined.

The volunteers were randomly assigned to three treatments, following a double-blind design. Treatment A was a placebo treatment (500 mg of maltodextrin per day); treatment B consisted in 140 mg of mango leaf extract (MLE; 60% mangiferin) and 50 mg of luteolin per day; and treatment C contained 140 mg of MLE (60% mangiferin), 600 mg of quercetin and 350 mg of tiger nut extract per day. The three treatments were divided in three daily doses administered every 8 h in methylcellulose capsules of identical appearance. The active ingredients in the extracts used in the test compositions is presented in Table 2.

TABLE 2

| | Magnifera indica L. extract | Arachis hypogaea extract | Sophora japonica extract | Cyperus esculentus extract |
|---|---|---|---|---|
| Part of the plant used | Leaves | Shell | Bulbs, skin | Dry tubers |
| Bioactive compounds | Mangiferin (≥60%) | Luteolin (≥90%) | [symbol] uercetin (≥90%) | HAF^{a} (≥5%) |
| (%, w/w) | | | | |
| | Hemomangiferin (≤2.5%) | | | Oleic acid glyceryl ester^{b} (2:1) |
| | | | | (≥91%) |
| | Isomangifein (trace levels) | | | Linoleic acid glyceryl ester^{b} |
| | | | | (≥7%) |
| | Sugars (≤10%) | | | S [symbol] (≥0.2%) |
| | | | | Myricetin^{b} (≥0.2%) |
| | | | | Sucrose (≤30%) |
| Moisture content | ≤7% | ≤7% | ≤7% | ≤7% |
| (%, w/w) | | | | |
| Botanical or native | Mangifera i. extract (100%) | Arachis h. extract (100%) | Sophora j. extract (100%) | Cyperus e. Extract (≥50%) |
| ingredient (%, w/w) | | | | |
| Non-botanical | None | None | None | Potato maltodextrin (≤25%) |
| ingredient (%, w/w) | | | | Arabic gum (≤25%) |

^{a}High Activity Fraction (HAF): Fraction [symbol] in ethyl acetate.
^{b}Relative to the amount of the HAF. [symbol]
indicates data missing or illegible when filed

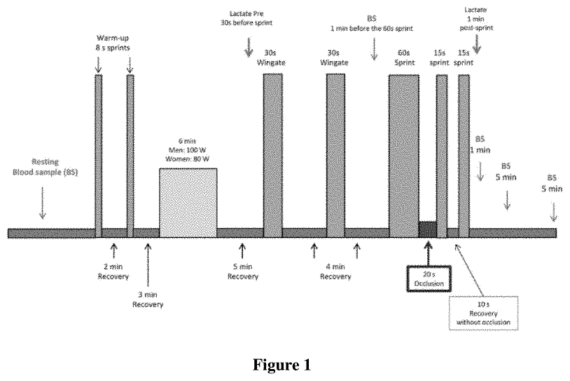

Subjects started supplement intake 48 h before the main experimental days. On the day of the experiment, subjects reported to the laboratory after a 10 h overnight fast, and 60 min before the start of the experiment ingested an additional dose of the supplement (i.e., ⅓ of the daily dose). Subjects were seated on cycle ergometers and performed two warming-up 8 s sprints in isokinetic mode at 80 rpm, separated by a 2 min interval (recovery phase 1) during which they pedaled with the cycle ergometer unloaded, as shown in FIG. 1. After a 3 min period of unloaded pedaling after the two warming-up sprints (recovery phase 2), the load was increased to 80 W in women, and 100 W in men for 6 min (80 rpm, ergometer set in rpm-independent mode). This was followed by unloaded pedaling for 5 min (recovery phase 3). After recovery phase 3, subjects stopped pedaling and the ergometer was switched to isokinetic mode. The subjects then performed a Wingate test (30 s all-out sprint in isokinetic mode at 80 rpm; Sprint 1). This was followed by another 3.5 min of unloaded pedaling and another 30 s period, during which they stopped pedaling and the ergometer was switched to the isokinetic mode (recovery phase 4). Then a second 30 s Wingate test was performed (Sprint 2), which was also followed by another 3.5 min of unloaded pedaling and another 30 s period of rest mode (recovery phase 5), also as shown in FIG. 1. Four min after the end of the second Wingate test, an all-out 60 s long sprint was carried out (Sprint 3). At the end of the 60 s sprint, the circulation of both lower extremities was instantaneously occluded for 20 s by inflating bilateral cuffs at 300 mm Hg. Cuffs were placed around the thighs during a preparation phase, as close as possible to the inguinal crease, and were connected to a rapid cuff inflator before they seated on the cycle ergometer. Ten seconds after the start of the occlusion, a reverse countdown was given and the subjects prompted to start pedaling again as fast and hard as possible, with the ergometer in isokinetic mode for 15 s (Sprint 4). At the start of the sprint, the cuff was deflated to allow full reestablishment of the circulation during the subsequent exercise. At the end of the 15 s sprint, the subjects pedaled slowly for another 5 s, and then stopped for 5 s to prepare for the final 15 s sprint (Sprint 5). During the 10 s of recovery that followed the 15 s post-ischemia sprint, as well as during the 15 s final sprint, the circulation was open. A capillary blood sample was drawn from the ear lobe to measure the concentration of lactate 1 min after the last sprint.

Blood samples were obtained from a heated hand vein at rest, 3 min after the second 30 s Wingate test, 1 min after the last sprint, and 5 and 10 min into the recovery period. The samples were analyzed for hemoglobin concentration, blood gases, electrolytes and acid-base balance.

B. Cerebral Oxygenation

Cerebral oxygenation was assessed at rest and during exercise using near-infrared spectroscopy, employing spatial resolved spectroscopy to obtain the tissue oxygenation index (TOI) using a path-length factor of 5.92. The NIRS optodes were placed on the right frontoparietal region at 3 cm from the midline and 2-3 cm above the supraorbital crest, to avoid the sagittal and frontal sinus areas. Using this optode placement the tissue oxygenation of the superficial frontal cerebral cortex was recorded. An additional optode was placed in the lateral aspect of the thigh at middle length between the patella and the anterosuperior iliac crest, over the middle portion of the Musculus vastus lateralis. The rate of muscle deoxygenation upon occlusion was calculated by determining the maximal slope of the linear decay of TOI over time. For this purpose, data were averaged every second and the slope TOI/time was calculated from the start of the occlusion to the end of occlusion, with a minimum interval of 4 s and a maximum of 20 s. Since the best linear fit was obtained with a 4 s interval, this was applied to all the occlusions.

C. Middle Cerebral Artery Blood Velocity

The mean blood velocity in the middle cerebral artery (MCAvₘₑₐₙ) was determined as an estimate of cerebral blood flow. Two Doppler 2 MHz transducers were applied bilaterally over the middle transtemporal window, and the readings from the transducers were averaged. A head harness was used to minimize potential movement artifacts. Resting cerebral oxygenation and MCAvₘₑₐₙ was calculated as the average of a 2 min collection period, while during exercise 5 s averages were generated and the average for the whole sprint reported.

D. Power Output

All pre-tests were performed on the same cycle ergometer, which maintains constant exercise intensity despite variations in pedaling rate. During all tests subjects were requested to maintain a pedaling rate close to 80 rpm. An isokinetic ergometer was used to determine power output. The ergometer was operated in a rpm-independent constant load during the warm-up and recovery phases, and switched to an isokinetic mode during the sprints, with the speed set at 80 rpm. During the isokinetic sprints, the subjects pedaled as fast and hard as possible, exerting as much force on the pedals as they could at each pedal stroke from the start to the end of the sprint. The servo-control brake system adjusted the braking force continuously to maintain a pedaling rate of 80 rpm during the entire sprint. Exhaustion was defined by the incapacity of the subject to maintain a pedaling rate above 50 rpm for 5 s, or by a sudden stop in pedaling.

D. Oxygen Demand and Deficit

The O₂ demand during the supramaximal exercise bouts was estimated from the linear relationship between the last min averaged VO₂ of each load, from 20 to 40 W to the highest intensity with an RER<1.00 in the incremental exercise test. The accumulated oxygen deficit (AOD), representing the difference between O₂ demand and VO₂, was determined.

E. Assessment of Pain and Effectiveness of Concealment

Subjects were requested to rate the level of pain felt during the occlusion from 0 to 10, 10 being the highest muscle pain ever suffered during or after exercise in their life. At the end of the experiment subjects were asked about the kind of supplement they suspected they had received to check on the effectiveness of concealment. After placebo administration, 7 out of 30 subjects guessed correctly that they had placebo. Following B supplementation, 11 subjects out of 30 guessed correctly that they had polyphenols, and after supplement C, 16 out of 30 guessed correctly that they had polyphenols. Subjects generally guessed that they had taken polyphenols when they felt better during the whole experiment.

F. Results

In this study, men and women had comparable levels of fitness. Men had a 15% greater VO₂max per kg of body mass than women, but the between-sex difference disappeared when the VO₂max was expressed per kg of lean mass of the lower extremities. Men had 41% greater anaerobic capacity than women per kg of body mass, but this difference was reduced to 23% when expressed in relation to the lean mass of the lower extremities. No significant between-sex differences were observed in the Wingate test when the values were normalized to the lean mass of the lower extremities.

1. Effects on Performance

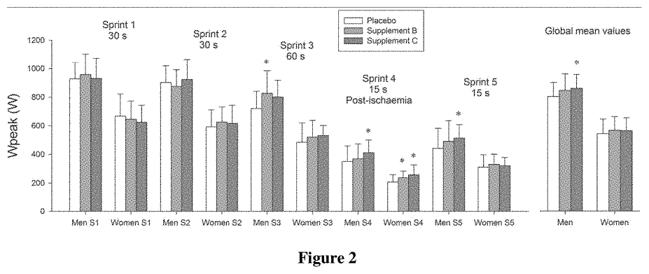

Supplements B (mangiferin+luteolin) and C (mangiferin+quercetin+tiger nut extract) enhanced performance during Sprint 3 (the 60-second sprint), relative to a placebo. Additionally, supplement C enhanced performance during Sprint 3 and Sprint 4 (the first 15-second sprint), relative to a placebo, and during Sprint 4, relative to Supplement B. The peak power output (Wpeak) observed during the sprints of FIG. 1 for the male and female subjects is recorded in FIG. 2 and Table 3. As seen in Table 3, Wpeak during Sprint 3 from patients administered placebo was 617.9 W, while mean power output (Wmean) during Sprint 3 was 233.4 W. In patients administered Supplement B, Wpeak increased to 695.1 W and Wmean increased to 247.8 W; these increases in power output were determined to be significant (p<0.05, compared with placebo). In patients administered Supplement C, Wpeak increased to 684.4 W and Wmean increased to 249.0 W; these increases in power output were also determined to be significant (p<0.05, compared with placebo). There was no significant difference during Sprint 3 between patients administered Supplement B and patients administered Supplement C.

TABLE 3

| | Sprint 1 | | | Sprint 2 | | | Sprint 3 | | |
|---|---|---|---|---|---|---|---|---|---|
| | W1A | W1B | W1C | W2A | W2B | W2C | W60A | W60B | W60C |
| Wpeak(W)^{b} | 814.9 | 822.8 | 798.0 | 768.1 | 767.7 | 7 [symbol] 0.0 | 617.9 | 695.1* | 684.4* |
| | 185.8 | 2 [symbol] 7.7 | 202.1 | 194.2 | 167.2 | 204.4 | 172.8 | 207.4 | 167.0 |
| Wmean(W)^{b} | 4 [symbol] 8.5 | 419.0 | 414. [symbol] | 390.4 | 383.7 | 3 [symbol] [symbol] .0 | 233.4 | 247.8* | 249.0* |
| | 10 [symbol] .3 | 98.8 | 98.3 | 94.5 | 9 [symbol] .1 | [symbol] 0.6 | 62.4 | 66.9 | 58.5 |
| HR (beat · min⁻¹) | 156.6 | 155.9 | 158.7 | 158.6 | 15 [symbol] .5 | 1 [symbol] 1.0 | 189.3 | 188. [symbol] | 170.1 |
| | 13.6 | 1 [symbol] .5 | 1 [symbol] .4 | 15.0 | 16.8 | 15.6 | 13. [symbol] | 14.5 | 11.4 |
| VO₂ (mL/min) | 100 [symbol] .3 | 1011.8 | 1010.4 | 10 [symbol] 5.7 | 1063.6 | 1 [symbol] 97.9 | 2415.1 | 2426.2 | 2429.1 |
| | 227.6 | 247.4 | 213.3 | 254.4 | 2 [symbol] [symbol] .9 | 236.4 | 57 [symbol] .8 | 504.8 | 5 [symbol] 7.0 |
| VCO₂ (mL/min) | 2012. [symbol] | 2023.7 | 2020. [symbol] | 2131.3 | 2127.3 | 211 [symbol] .7 | 2415.1 | 2426.2 | 2429.1 |
| | 455.2 | 49 [symbol] .7 | 42 [symbol] .7 | 508.8 | 511.7 | 472.9 | 578.8 | [symbol] 04. [symbol] | 507.0 |
| O₂ deficit (mL) | 1611.5 | 1601.0 | 1605. [symbol] | 1 [symbol] 67.1 | 1 [symbol] 78.4 | 1 [symbol] 32.3 | 810.0 | 927.2 | 768.1 |
| | 520. [symbol] | 536.4 | 4 [symbol] 5.1 | 4 [symbol] 6.8 | 445. [symbol] | 440.2 | 427.2 | 531.7 | 432.3 |
| RER | 1.02 | 1.04 | 1.02 | 1.03 | 1.04 | 1.03 | 0.97 | 0.98 | 0.9 [symbol] |
| | 0.11 | 0.11 | 0.11 | 0.08 | 0. [symbol] 7 | 0.0 [symbol] | 0.07 | 0.07 | 0.05 |
| VE (L/min) | 78.2 | 79.2 | 76.9 | 9 [symbol] .9 | 99. [symbol] | 98. [symbol] | 112.2 | 113.2 | 111.0 |
| | 22. [symbol] | 22.1 | 2 [symbol] .1 | 28.7 | 2 [symbol] .7 | 28.0 | 29.4 | 30.3 | [symbol] 0.5 |
| P_{ET}O₂ (mmHg) | 11 [symbol] .5 | 114. [symbol] | 112. [symbol] | 119.7 | 119.7 | 119.3 | 119. [symbol] | 120.1 | 120.0 |
| | 5. [symbol] | 4.5 | 11. [symbol] | 3.2 | 3.1 | 4.7 | 2.9 | 2.6 | 2.8 |
| P_{ET}CO₂ (mmHg) | 29.0 | 30.5 | 2 [symbol] .9 | 25.6 | 2 [symbol] .9 | 26.3 | 24.2 | 24.1 | 24.4 |
| | 3.7 | 3.7 | 4.3 | 2.3 | 2.5 | 3.5 | 2.5 | 2.2 | 2.5 |

| | Sprint 4 | | | Sprint 5 | | |
|---|---|---|---|---|---|---|
| | W15A | W15B | W15C | W15FA | W15FB | W15FC |
| Wpeak(W)^{b} | 288.0 | 311.9 | 343.9*¶ | 385.3 | 421.0 | 430.4* |
| | 113.3 | 106.1 | 11.2 | 135.0 | 142.0 | 12 [symbol] .3 |
| Wmean(W)^{b} | 165.4 | 172.5 | 18 [symbol] . [symbol] *¶ | 201.3 | 207.7 | 209.5 |
| | [symbol] 5.8 | 53.2 | 5 [symbol] . [symbol] | 62.5 | 51.7 | 50.4 |
| HR (beat · min⁻¹) | 173.3 | 174.9 | 176.2 | 173.3 | 175.0 | 178.1 |
| | 1 [symbol] .9 | 14.2 | 9.0 | 15.7 | 18.2 | 10.1 |
| VO₂ (mL/min) | 54 [symbol] .1 | 549.2 | 550.7 | 635.7 | 641.9 | 644.4 |
| | 160.9 | 161.7 | 131.9 | 185. [symbol] | 181. [symbol] | 193.0 |
| VCO₂ (mL/min) | 2180.3 | 2197.0 | 2202.8 | 2542.8 | 2587.8 | 2577. [symbol] |
| | 643.7 | 648.7 | 527.7 | 741.9 | 725.8 | 772.2 |
| O₂ deficit (mL) | 57.0 | 75.9 | 90.8* | 63.5 | 70.2 | 76.2 |
| | 94. [symbol] | 142.9 | 10 [symbol] .5 | 115.7 | 120.7 | 123.4 |
| RER | 1.18 | 1.18 | 1.16 | 1.04 | 1.0 [symbol] | 1.07 |
| | 0.09 | 0.0 [symbol] | 0.07 | 0.08 | 0.06 | 0.12 |
| V_{E} (L/min) | 118.8 | 121.9 | 117.1 | 121.1 | 122.5 | 123.3 |
| | [symbol] 5.0 | 33.4 | 37.0 | [symbol] [symbol] .7 | 35.3 | 36. [symbol] |
| P_{ET}O₂ (mmHg) | 122.6 | 122.9 | 119.5 | 119.9 | 119.9 | 12 [symbol] .5 |
| | 2.6 | 2.4 | 17.1 | 2.4 | 2.5 | 2.7 |
| P_{ET}CO₂ (mmHg) | 24.6 | 24.7 | 24.3 | [symbol] 5.7 | 25.7 | 2 [symbol] .7 |
| | 2.6 | 2.2 | 4.5 | 2.5 | 2.5 | 2.7 |

Wpeak, peak power output; Wmean, mean power output; HR, heart rate; VO₂, oxygen uptake; VCO₂, CO₂ production; RER, respiratory exchange ratio; V_{E}, pulmonary ventilation; P_{ET}O₂, end-tidal O₂ pressure; P_{ET}CO₂, end-tidal CO ₂ pressure; W1, first Wingate (30 s sprint); W2, second Wingate (30 s sprint); W60, 60 s sprint; W15, 15 s sprint post-ischemia; W15F, final 15 s sprint; Treat, treatment effect. A, Placebo; B, luteolin + Mangiferin; C, Mangiferin + Quercetin + Tiger nut extract.
*P < 0.05 compared with placebo;
¶P < 0.05 compared with treatment B. [symbol]
indicates data missing or illegible when filed

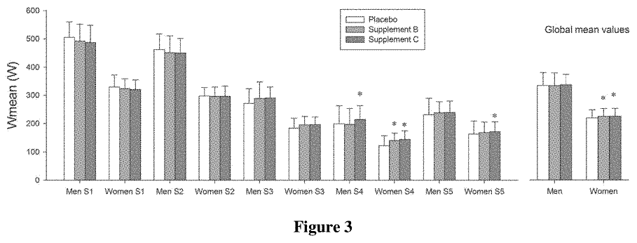

Also as seen in Table 3, Wpeak during Sprint 4 (the first 15-second sprint) from patients administered placebo was 288.0 W, while Wmean during Sprint 4 was 165.4 W. In patients administered Supplement B, Wpeak increased to 311.9 W and Wmean increased to 172.5 W; however, these increases were not significant, relative to placebo. In patients administered Supplement C, Wpeak increased to 343.9 W and Wmean increased to 183.9 W. The increases in power output in patients administered Supplement C during Sprint 4 were determined to be significant, relative to placebo (p<0.05). Also, the differences in both peak and mean power output between patients administered Supplement B and patients administered Supplement C were determined to be significant (p<0.05). During Sprint 5, patients administered Supplement C also showed a statistically significant increase in peak power output relative to patients administered placebo (p<0.05). During the 60 s long sprint, supplements B and C increased Wpeak by 12.5 and 10.8%, respectively. In Sprint 4, performed after ischemia, supplement C increased Wpeak by 19.4% compared to placebo (p<0.001) and by 10.2% compared to supplement B (p<0.05). The total amount of work performed was 2.4% higher following the ingestion of supplements B and C, compared with placebo in women (34.1±4.3, 34.9±4.1, and 34.9±4.0 kJ, for placebo and supplements B and C, respectively, p<0.05). The corresponding values in men were 51.7±6.7, 52.1±7.3, and 52.3±5.8 kJ, respectively (p>0.3). During the sprint performed after ischemia (Sprint 4), supplement C enhanced Wmean by 11.2% (p<0.001) compared with the placebo trial and 6.7% compared with supplement B (p=0.012). As seen in FIG. 3, Supplement B significantly increased mean power output during Sprint 4 for women. Supplement C significantly increased mean power output during Sprint 4 for both men and women.

2. Pulmonary Gas Exchange

In women, the peak VO₂ reached during the repeated sprints was 5.8% greater after the administration of supplements (mean of both trials) compared with the placebo trial (2,189±334 and 2,316±403 mL/min, for placebo and supplements, respectively, P=0.012). No such effect was observed in men (3,265±406 and 3,318±422 mL/min, placebo and supplements, respectively, P=0.42). The O₂ deficit incurred was 2.7-fold greater after the ingestion of supplement C than after placebo in men (P=0.001), while it remained at the same level in women.

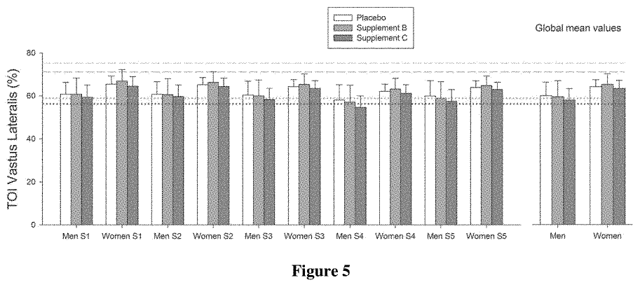

Neither the accumulated VO₂ nor the O₂ deficit observed during the sprints were significantly altered by any of the treatments, when all sprints were analyzed conjointly. Nevertheless, during the 15 s sprint performed after ischemia (Sprint 4), the vastus lateralis oxygenation index tended to be a slightly lower value after the administration of supplement C, compared with placebo (P=0.056), as shown in FIG. 5.

3. Brain Oxygenation

Resting brain oxygenation was lower in women than in men (P<0.001). This was associated with lower P_{ET}CO₂ (end tidal CO₂ concentration, mm Hg) in women than in men (30.7±2.6 and 34.2±2.1 mm Hg in women and men, respectively, P<0.001). In women, both supplements increased frontal lobe oxygenation at rest (59.4±5.7, 64.9±3 0.8, and 64.9±6.4%, for placebo, Supplement B, and Supplement C, respectively, P<0.05, for the comparisons of supplement B and C against placebo). In men, brain oxygenation remained substantially unchanged (69.3±5.4, 69.1±4.2, and 68.0±4.4%, for placebo, Supplement B, and Supplement C, respectively, P>0.50, for the comparisons of supplement B and C against placebo).

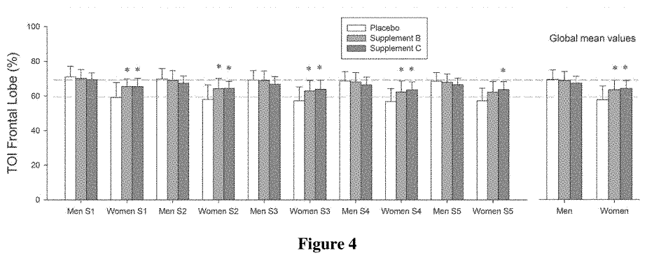

In women, brain oxygenation during the sprints was greater after the ingestion of supplements B and C than placebo (FIG. 4). Likewise, during the 20 s ischemic recovery that followed the 60 s long sprint (sprint 3), brain oxygenation was higher after the ingestion of supplements B and C in women than in men (57.7±7.2, 63.1±6.0, and 64.0±4.8%, for placebo, and supplements B and C, respectively, P<0.05; for the comparison of supplement B and C with placebo, P=0.05). The corresponding values in men were not significantly altered by the ingestion of supplements (68.0±3.8, 67.9±5.7, and 66.3±4.3%, for placebo, and supplements B and C, respectively; for the comparison of supplement B and C with placebo, P>0.30).

### Example 2. Studies on Human Patients: Experimental Procedure

A. Subjects

Twelve healthy male physical education students (age=21.3±2.1 yr, height=176.6±5.8 cm, body mass=75.7±9.9 kg, body fat=20.3±5.3%, VO2max: 3.69±0.47 L/min and 49.4±8.2 mL/(Kg·min)) agreed to participate in this investigation (Table 1). Before volunteering, subjects received full oral and written information about the experiments and possible risks associated with participation. Written consent was obtained from each subject. The study was performed by the Helsinki Declaration and approved by the Ethical Committee of the University of Las Palmas de Gran Canaria (CEIH-2016-02). The sample size required to allow detecting a 5% improvement of performance with a statistical power of 0.8 (α=0.05), assuming a coefficient of variation for the ergometric test below 5%, was eight subjects. To account for potential dropouts twelve subjects were finally recruited.

### General Procedures

The inclusion criteria for participation in the study were: age from 18 to 35 years old; male without chronic diseases or recent surgery; non-smoker; normal resting electrocardiogram; body mass index below 30 and above 18; no history of disease requiring medical treatments lasting more than 15 days during the preceding 6 months; no medical contraindications to exercise testing; and lack of allergies to peanuts or mango fruit. All volunteers applying met the inclusion criteria.

After inclusion, a medical history, resting electrocardiogram, a blood analysis including the assessment of a basic hemogram and general biochemistry, and a basic urine analysis were carried out to verify the health status of participants. Then subjects were assigned to a control placebo trial or to a treatment trial with a double-blind crossover design. Six subjects were randomly allocated to a placebo (P) and another six to a treatment group (T). The placebo received microcrystalline cellulose capsules containing 500 mg of maltodextrin, while the treatment group received similar capsules containing luteolin and mangiferin. The daily doses were for three subjects (50 mg of luteolin and 100 mg Mangiferin; low dose treatment group; LT) and for the other three (100 mg of luteolin and 300 mg Mangiferin; low dose treatment group; LT). Subjects ingested the supplements every three hours during fifteen days, then after 4-6 weeks, treatment groups received placebo, and the placebo group was again split into low and a high dose treatment groups, also for 15 days.

Forty-eight hours after the start of the supplementation subjects reported to the laboratory early in the morning under fasting conditions and received an extra dose of the assigned supplements. After that, their body composition was determined using dual-energy x-ray absorptiometry (Lunar iDXA, General Electric, Wisconsin, USA), followed by the assessment of their resting metabolic rate (BMR). Then near-infrared spectroscopy (NIRS) optodes were placed on the frontal lobe and vastus lateralis as previously reported (Curtelin D, Morales-Alamo et al. J Cereb Blood Flow Metab 271678X17691986, 2017). With the subjects resting supine a 10 cm wide cuff connected to a fast compressor (SCD10, Hokanson, Bellevue, USA) was placed around the right thigh, as proximal as possible and the leg elevated for 3 min. At the end of the three min, the circulation was occluded for 8 min, and the cuff was released and the hyperemic response measured during the next two minutes. After that, a forearm vein was catheterized and a resting blood sample obtained before the start of the exercise protocol.

### Exercise Protocol

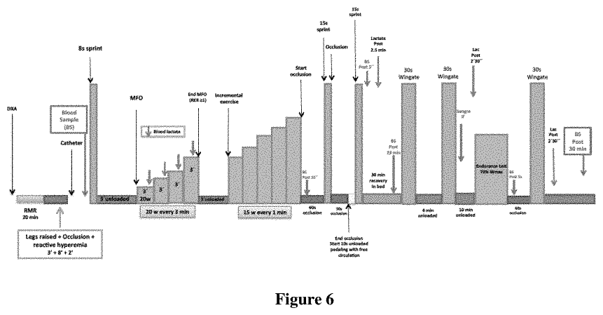

The exercise protocol started with an 8 s isokinetic sprint on a cycle ergometer (Excalibur Sport 925900, Lode, Groningen, The Netherlands) (FIG. 6). This sprint was used as a control to obtain the instantaneous peak power output (Wpeak-i) under rested conditions. This was followed by a recovery period during which the subjects pedaled at low speed (˜40 rpm) with no load. Next, an incremental test was applied to determine the maximal fat oxidation capacity (MFO) (see below). The MFO test was followed by two min of unloaded pedaling, and then the load was increased to the same level reached at the end of the MFO test and increased 15 W every min until exhaustion to determine the VO2max. Immediately, upon exhaustion, the cuffs were instantaneously inflated at maximal speed and pressure (i.e., 300 mmHg) to completely occlude the circulation (ischemia), as previously reported (Morales-Alamo D, Losa-Reyna et al. J Appl Physiol (1985) 113: 917-928, 2012). After 10 s of the start of the ischemia, a blood sample was obtained from the forearm vein. The subjects remain seated on the bike quiet and without pedaling during the ischemia period. After 60 s the occlusion was instantaneously released and the subjects requested to sprint as fast and hard as possible during 15 s. At the end of the sprint, a second occlusion was started for 30 s, which was followed by 10 s of free circulation and the subjects got ready for another 15 s sprint, which was carried out after a cycle of ischemia (30 s) and 10 s (reperfusion). Five seconds after the end of this sprint, a blood sample was obtained from the forearm, and at 2.5 min another blood sample was obtained from the hiperemized earlobe to measure blood lactate concentration (Lactate Pro, Akray) and the subjects were allowed to rest for 30 min. The first 20 min they rested lying on a bed and the last 10 cycling at low speed on the cycle ergometer. At the 29th min of this recovery period, a blood sample was obtained. At the completion of the recovery, a Wingate test (sprint lasting 30 s) was performed followed by a four min recovery period with the subjects pedaling a low speed with the cycle ergometer unloaded. At the end of this short recovery, a second Wingate test was performed. The second Wingate was followed by a 10 min recovery with the subjects pedaling at slow speed with the cycler ergometer unloaded. At 2.5th min, a blood sample was obtained from the hiperemized earlobe to measure blood lactate concentration, followed by a forearm blood sample at the 9th min of this recovery period. At the completion of the 10 min recovery period, a submaximal constant intensity time trial to exhaustion was started at 70% of the intensity reached at exhaustion in the incremental exercise test used to measure VO2max, or Wmax intensity. In control experiments our subjects were able to sustain this intensity for 20-60 min in rested conditions, depending on their fitness status. This test was used to assess the effects of endurance capacity, since the test starts with very low glycogen levels, replicating the conditions of the final kilometers of a typical endurance competition. At the exhaustion, the circulation of both legs was occluded again for 60 s. A blood sample was obtained from the forearm vein at the beginning of the occlusion (5th s). At the 60th s the occlusion was instantaneously released, and the subjects requested to perform a final Wingate (30 s) sprint. At the end of this sprint, the subjects remained seated on the bike while pedaling a low speed with the cycle ergometer unloaded. After 2.5 min another blood sample was obtained from the hiperemized earlobe to measure blood lactate. Then the subjects moved to a bed and rested until the 30th min from the end of the last Wingate test, and at this time point the final blood sample was obtained (FIG. 6).

This exercise protocol was repeated after 15 days of supplementation, to determine potential effects due to chronic supplementation. After 4-6 weeks, the acute and chronic phase was repeated following the crossover design described above.

### Power Output and VO₂Max

Power output during the sprint is reported as instantaneous peak power (Wpeak-i), and as the mean power output achieved during the full duration of the sprints (Wmean-8 and Wmean 30). Oxygen uptake was measured with a calibrated metabolic cart (Vyntus; Carefusion-BD, Madrid, Hospital Hispania). Respiratory variables were analyzed breath-by-breath and averaged every 5 s during the sprints. During maximal exercise 15 breath rolling average was generated starting from 120 s before the end of exercise and highest 15-breath averaged value was taken as the VO2max.

Cerebral Oxygenation and Musculus vastus lateralis Oxygenation

Cerebral oxygenation was assessed using near-infrared spectroscopy (NIRS, NIRO-200, Hamamatsu, Japan) employing spatial resolved spectroscopy to obtain the tissue oxygenation index (TOI) using a pathlength factor of 5.92 (Van der Zee P et al. Adv Exp Med Biol 316: 143-153, 1992.). The NIRS optodes were placed on the right frontoparietal region at 3 cm from the midline and 2-3 cm above the supraorbital crest, to avoid the sagittal and frontal sinus areas. With this optode placement, the tissue oxygenation of the superficial frontal cerebral cortex is recorded. This region is irrigated by the anterior cerebral artery, which, like the MCA, receives its flow from the internal carotid artery. Both MCA and anterior cerebral arteries communicate through the circle of Willis. An additional optode was placed in the lateral aspect of the thigh at middle length between the patella and the anterosuperior iliac crest, over the middle portion of the m. vastus lateralis.

### Maximal Fat Oxidation

This test was performed in the same cycler ergometer started at 20 W, and the load was increased by 20 W every 3 min (1, 69). The arm cranking MFO test began at 10 W for 5 min followed by a 10-W increase every 3 min. The leg MFO test started at 30 W for 5 min, followed by a 30-W increment every 3 min. At the end of the 3-min period during which the subject exhibited an RER>1.0, the exercise was stopped.

### Exercise Efficiency, Supramaximal Exercise O₂ Demand and Oxygen Deficit

The O₂ demand during the sprints was calculated from the linear relationship between the last 60-s averaged VO₂ of each load, measured during the MFO. The accumulated oxygen deficit (AOD), representing the difference between O₂ demand and VO₂, was determined as previously reported (Calbet J A, Chavarren J, and Dorado C. Eur J Appl Physiol 76: 308-313, 1997., Dorado C, Sanchis-Moysi J, and Calbet J A Can J. Appl Physiol 29: 227-244, 2004). The energy efficiency of exercise was determined as previously reported (Chavarren J, and Calbet J A. Eur J Appl Physiol 80: 555-563, 1999), using the data collected during the MFO tests.

### Diet Analysis

Dietary information was collected from all subjects before the start of the supplementation, and after one week into each supplementation period using dietary logs including four days. For this purpose, subjects were provided with a dietary diary and a kitchen scale (1 g precision from 0 to 5000 g, calibrated in our laboratory with Class M1 calibration weight, Schenk) and instructions to report in grams all food and drinks ingested. The information recorded was later analyzed with specific software for the Spanish diet (Dial, Alce Ingenieria, Madrid, Spain (Ortega R M. et al. Eur J Clin Nutr 61: 77-82, 2007).

### Blood Samples

Ten mL blood samples were obtained at each sampling point and processed to obtain serum and plasma, and immediately frozen at −80° C. Further analysis will be carried out on this samples including the concentration of carbonylated proteins as a marker for oxidative stress using the“OxyBlot” protein oxidation kit (Intergen Company, Purchase, N.Y.) as previously described (Morales-Alamo D et al. J Appl Physiol 113: 917-928, 2012, Romagnoli M et al. Free Radic Biol Med 49: 171-177, 2010).

Statistics

Variables were checked for normal distribution by using the Shapiro-Wilks test. When necessary, the analysis was carried out on logarithmically transformed data. A double repeated-measures ANOVA test with time (two levels: acute and chronic) and treatment with another two levels (Placebo vs. treatment) was first applied. Pairwise comparisons were carried using the least significant post hoc test (LSD). A comparison between high and low dose was also carried out using a repeated measures analysis with dose levels as between-subjects factor with two levels (low and high). The relationship between variables was determined using linear regression analysis. Values are reported as the mean±standard error of the mean (unless otherwise stated). P 0.05 was considered significant. Statistical analysis was performed using SPSS v.15.0 for Windows (SPSS Inc., Chicago, Ill.).

### Results

Polyphenols had no significant effects on the hemogram and blood biochemistry clinical tests. The diet was not significantly altered by the treatment regarding total energy, macronutrients, vitamins, dietary fiber and plant sterols intakes. Likewise, no significant alterations were observed in resting blood lactate concentration, resting metabolic rate or the body composition. Nevertheless, the resting breathing frequency and the resting P_{ET}CO₂ were slightly increased and decreased, respectively from the first to the second assessment (Table 1). The resting blood pressure, blood lactate concentration and heart rate were not altered by the intervention.

### Incremental Exercise Test

All respiratory variables responded similarly to the placebo and the polyphenol treatment. Indices of maximality of tests were also similar, indicating that the subjects exercised to a similar extent in all tests. Neither the VO₂max nor the load reached at exhaustion (Wmax) were affected by the treatment. There was a small 2 mmHg improvement in PETO2 in second test which was also accompanied by a small reduction in PETCO2 (˜2 mmHg).

Lactate responses to submaximal exercise were almost identical. Although, blood lactate concentration at 200 W was 11% lower after the polyphenol treatment, this effect did not reach statistical significance (P=0.11). Delta efficiency was transiently improved 48 h after the start of polyphenols in the group receiving the lower dose (P=0.002, compared to placebo; Treatment×time×dose interaction P=0.001) (Table 1). This effect evanished following 15 days of supplementation (P=0.87 compared to placebo). Polyphenols supplementation did not alter the MFO (Table 1) nor peak HR.

Sprint Exercise after Ischemia/Reperfusion

The PPOᵢ was not altered by the acute administration of polyphenols (FIG. 2A). Following fifteen days of supplementation, PPOᵢ in the sprints preceded by ischemia was 500.0±120.1 and 566.4±141.9 W, in the placebo and polyphenol trial respectively, P=0.11). Nevertheless, from the first (48 h) to second trial (15 days), PPOi was enhanced by 22% when the subjects were taken polyphenols (P<0.05), being this effect more marked in the first (+31%) than second sprint (+14%) (first sprint compared with the second sprint, P<0.05) (ANOVA sprint×trial×treatment×dose interaction P=0.026). There were not significant differences between the higher and lower doses of polyphenols on PPOᵢ.

In the sprints post ischemia performed with polyphenols, the mean power output developed during the first 5 s was increased by 23% from 48 h (272.5±63.8 W) to 15 days (333.8±93.2 W) (P=0.01). In contrast, no significant changes were observed form 48 h to 15 days in the placebo conditions (FIG. 2B). The peak blood lactate measured 2.5 min after the last sprint postischemia was unchanged in the placebo experiments (9.8±2.7 and 10.4±2.1 mM, P=0.35), but increased from 9.5±2.5 to 11.4±1.8 mM (48 h and 15 days, respectively) after the ingestion of polyphenols (P=0.04; time×treatment interaction P=0.29).

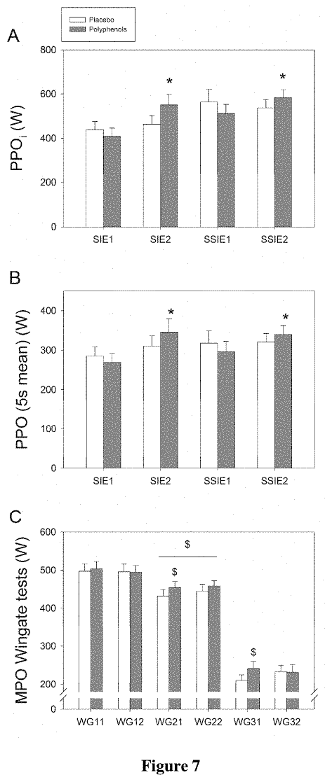

(FIG. 7)

### Wingate Tests

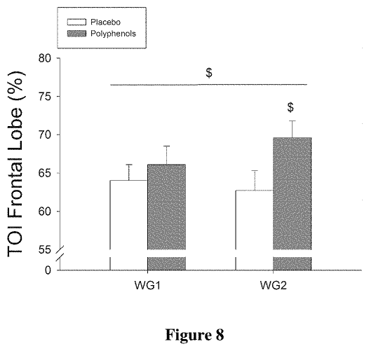

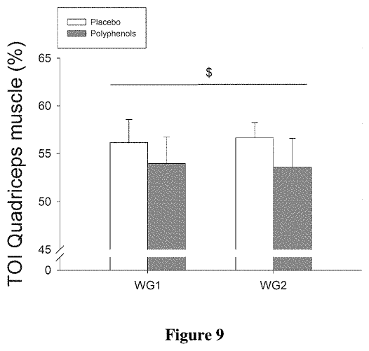

Compared to placebo, polyphenol intake resulted in 4.0% greater MPO (acute and chronic assessments combined, P=0.017; ANOVA Wingate×time×treatment P=0.027). Acutely, compared to placebo, polyphenol administration enhanced MPO by 5% in the second Wingate test (P=0.009) (FIG. 7C). This was accompanied by enhanced brain oxygenation (FIG. 8) (Treatment effect P=0.02), being this response greater for the higher dose (Treatment×dose interaction P=0.047). Quadriceps muscle oxygenation index during sprint exercise was significantly lower after the ingestion of polyphenols, both after 48 h (59.7±6.0 and 57.9±6.4%, P=0.007) and 15 days (60.1±3.9 and 57.0±6.1%, P=0.007) supplementation (Treatment×dose interaction P=0.01) (FIG. 9).

The last sprint was performed after a time trial to exhaustion followed by a 60 s ischemia, in a situation of extreme fatigue and exhaustion of the energy resources. After 48 h of supplementation, MPO was 15% higher in the group receiving polyphenols than in the placebo group (P=0.04). No significant differences were observed neither in brain oxygenation index during the last Wingate test (65.8±8.6 and 68.5±7.2%, for the placebo and polyphenols trials, respectively, P=0.38) nor in quadriceps muscle oxygenation index (57.1±6.7 and 55.8±9.0%, for the placebo and polyphenols trials, respectively, P=0.22).

No significant differences were observed in the mean lactate responses after incremental exercise and the three Wingate tests (10.3±2.4 and 11.1±2.3 mM, for the placebo and polyphenols trials, respectively, P=0.15).

### Final Time Trial

No significant effects were observed in the total work performed during the final time trial (101.3±56.6±103.5±61.6 Kj, for the placebo and polyphenol trial, respectively P=0.85). Neither brain oxygenation index (64.6±6.5 and 68.0±6.0%, for the placebo and polyphenol trial, respectively P=0.18) nor quadriceps muscle oxygenation index (61.3±6.3 and 60.6±8.5%, for the placebo and polyphenol trial, respectively P=0.34).

### Quadriceps Muscle O₂ Extraction During Ischemia

During the first five seconds of the occlusion quadriceps muscle oxygenation index was reduced to lower levels after the ingestion of polyphenols (P=0.04, FIG. 9).

This example shows that the combination of a mango leaves extract rich in mangiferin with luteolin enhances exercise performance during sprint exercise and facilitates muscle oxygen extraction in the fatigued state. In addition, this polyphenolic combination improves muscle performance after ischemia/reperfusion by two main mechanisms. Firstly, it facilitates muscle oxygen extraction as demonstrated by the greatest reduction of the muscle oxygenation index during the first five seconds of total occlusion of the circulation. Secondly, it may facilitate the production of ATP through an additional recruitment of the glycolysis, as indicated by the greater levels of blood lactate concentration observed in the sprints performed after ischemia/reperfusion. Importantly, MLE and luteolin enhanced mean power output during prolonged sprints (30 s Wingate test) carried out after 30 min of recovery. This improvement in prolonged sprint performance was accompanied by better brain oxygenation and larger muscle oxygen extraction during the sprints.

A mango leaves extract combined with luteolin improves muscle O₂ extraction. In the present example we have shown that MLE+Luteolin supplementation allows the skeletal muscle to reach lower levels of tissue oxygenation. This effect could be explained by a reduction of skeletal muscle O₂ delivery, better microvascular distribution of perfusion (prioritizing the active skeletal muscle fibers) and enhanced mitochondrial O₂ extraction. Since the effect of MLE+Luteolin was greater during the second Wingate test, i.e., when skeletal muscle blood flow is expected to increase quicker a to a higher level, a reduction in O₂ delivery to exercising muscles is unlikely. Moreover, the fact that the HR response was not different with supplementation also argues against a different cardiovascular regulation between conditions. The matching between perfusion and VO2 at the microvascular level second explanation cannot be tested with current technology during whole body exercise in humans. Thus, the most plausible mechanism by which polyphenol supplementation could have enhanced O₂ extraction is by improving mitochondrial respiration, which could be impaired by the high levels of reactive oxygen and nitrogen species (RONS) produced during repeated sprint exercise.

A mango leaves extract combined with luteolin enhances sprint performance after ischemia/reperfusion. Sprint performance after ischemia reperfusion was improved, particularly after the first ischemia, which was followed immediately by a sprint, while the effect was less marked during the second 15 s sprint, which was preceded by 30 s of ischemia and 10 s of active recovery with reoxygenation. In the present investigation the inhibitory action of MLE+luteolin on XO might have been beneficial during high intensity-exercise, ischemia and ischemia/reperfusion by reducing superoxide and secondary RONS production and attenuating NO production from nitrite and, hence, the inhibition of mitochondrial respiration. Consequently, MLE+luteolin could have facilitated mitochondrial respiration and aerobic energy production during the sprints and ischemia periods, as actually shown by the lower levels of muscle oxygenation observed in this investigation when the experiments were preceded by the ingestion polyphenols.

Mangiferin administration combined with Luteolin increases frontal lobe oxygenation during repeated sprint exercise.

Better frontal lobe oxygenation was observed during the prolonged sprints performed after the ingestion of MLE+luteolin. These effects may be related to a better distribution of blood flow between tissues or enhanced cerebral vasodilation facilitated by the polyphenols.

In summary, supplementation with mango leaves extract combined with luteolin enhances exercise sprint performance, likely by improving brain oxygenation and enhancing muscle oxygen extraction.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

1. A method for increasing power output, brain frontal lobe oxygenation, peak oxygen consumption, or mitigating the effects of ischemia/reperfusion injury, of an athlete, sports person or exerciser, comprising administering a formulation to said athlete, sports person or exerciser, said formulation comprising:
a. an effective amount of mangiferin; in combination with an effective amount of an active ingredient selected from the group consisting of luteolin, quercetin, an ethyl acetate extract of Cyperus esculentus tubers, and mixtures thereof; and
b. optionally, a carrier.

2. The method according to claim 1, wherein said formulation comprises:
from 5 parts to 1000 parts/day of mangiferin; in combination with either:
from 2 to 1000 parts of luteolin,
from 20 to 2000 parts of quercetin,
from 1 to 1000 parts of an ethyl acetate extract of Cyperus esculentus tubers, or
with mixtures thereof; and
optionally, a carrier.

3. The method according to claim 1, wherein said formulation comprises:
from 50 mg to 5,000 mg/day of mangiferin; in combination with either:
from 10 mg/day to 5,000 mg/day of luteolin;
from 100 mg/day to 10 g/day quercetin;
from 50 mg/day to 5,000 mg/day of an ethyl acetate extract of Cyperus esculentus tubers;
or with mixtures thereof; and
optionally, a carrier.

4. The method according to claim 1, wherein said athlete, sports person or exerciser is a woman.

5. The method according to claim 1, wherein it is for increasing power output by said athlete, sports person or exerciser, wherein power output is measured in terms of peak power output or mean power output.

6. The method according to claim 1, wherein it is for increasing brain frontal lobe oxygenation or peak oxygen consumption in a female athlete, sports person or exerciser.

7. The method according to claim 1, wherein it is for mitigating the effects of ischemia/reperfusion injury.

8. The method of claim 1, wherein said formulation comprises from 50 mg/day to 5,000 mg/day of mangiferin and from 10 mg/day to 5,000 mg/day of luteolin.

9. The method according to claim 1, wherein the formulation comprises from 50 mg/day to 5,000 mg/day of mangiferin; from 100 mg/day to 10 g/day quercetin; and from 5 mg/day to 5,000 mg/day of an ethyl acetate extract of Cyperus esculentus tubers.

10. The method according to claim 1, wherein said mangiferin is a component of an extract of Mangifera indica, said extract of Mangifera indica comprising from 10% to 90% by weight of mangiferin

11. The method according to claim 1, wherein said luteolin is a component of an extract of Arachis hypogaea, said extract of Arachis hypogaea comprising from 50% to 95% by weight of luteolin.

12. The method according to claim 1, wherein:
said quercetin is a component of an extract of Sophora japonica, said extract of Sophora japonica comprising from 50% to 95% by weight of quercetin; and
said ethyl acetate extract of Cyperus esculentus tubers comprises:
oleic acid glyceryl esters, linoleic acid glyceryl esters, or a combination thereof;
phytosterols;
flavonoids; and
mixtures thereof.

13. The method according to claim 1, wherein the method comprises administration of said composition in a single dosage form for once-daily administration.

14. The method according to claim 1, wherein the method comprises administration of said composition in multiple dosage forms.

15. The method according to claim 14, wherein said multiple dosage forms are such that a first dosage form comprises mangiferin and a second dosage form comprises luteolin, quercetin, an ethyl acetate extract of Cyperus esculentus tubers, or a mixture thereof.

16. The method according to claim 7, wherein it is for mitigating the effects of ischemia/reperfusion injury in skeletal muscle.

17-29. (canceled)
